SEQUENCE LISTING (4 sequences)

SEQ ID NO: 1 - protein, 431 aa, Homo sapiens [sequence omitted]

SEQ ID NO: 2 - protein, 411 aa, Homo sapiens
SNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 3 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 4 - protein, 532 aa, Homo sapiens
RRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

(19) United States
(12) Patent Application Publication
(10) Pub. No.: US 2020/0046811 A1
(43) Pub. Date: Feb. 13, 2020

(54) METHODS AND COMPOSITIONS FOR MODULATING FGF23 LEVELS

(71) Applicant: Northwestern University, Evanston, IL (US)

(72) Inventors: Douglas E. Vaughan, Chicago, IL (US); Mesut Eren, Harwood Heights, IL (US); Aaron T. Place, Chicago, IL (US); Toshio Miyata, Evanston, IL (US)

(21) Appl. No.: 16/360,244

(22) Filed: Mar. 21, 2019

Related U.S. Application Data
(63) Continuation of application No. 14/949,266, filed on Nov. 23, 2015, now abandoned.
(60) Provisional application No. 62/082,969, filed on Nov. 21, 2014.

(51) Int. Cl. A61K 38/48; A61K 31/341; A61K 31/381; A61K 38/49; A61K 31/495; A61K 45/06

(52) U.S. Cl. CPC A61K 38/482; A61K 31/341; A61K 31/381; A61K 38/49; G01N 2800/52; A61K 45/06; C12Y 304/21073; C12Y 304/21068; G01N 2333/50; A61K 31/495

(57) ABSTRACT

The present invention provides compositions, systems, and methods for treating a condition characterized by elevated Fibroblast Growth Factor 23 (FGF23) with a composition comprising: i) an agent that causes an increase in expression of urokinase plasminogen activator (uPA) and/or tissue plasminogen activator (tPA), ii) purified uPA, and/or purified tPA.

The present application is a continuation of U.S. patent application Ser. No. 14/949,266, filed Nov. 23, 2015, which claims priority to U.S. Provisional Application Ser. No. 62/082,969 filed Nov. 21, 2014, each of which is herein incorporated by reference in its entirety.

### FIELD OF THE INVENTION

The present invention provides compositions, systems, and methods for treating a condition characterized by elevated Fibroblast Growth Factor 23 (FGF23) with a composition comprising: i) an agent that causes an increase in expression of urokinase plasminogen activator (uPA) and/or tissue plasminogen activator (tPA), ii) purified uPA, and/or purified tPA.

### BACKGROUND

Elevated plasma levels of Fibroblast Growth Factor 23 (FGF23) are present in a variety of human diseases and conditions including chronic kidney disease, left ventricular hypertrophy and congestive heart failure, autosomal dominant hypophosphatemic ricketts, osteomalacia, Vitamin D deficiency, fibrous dysplasia, and aging. FGF23 is synthesized in bone by osteogenic cells, and is secreted into the circulation where it primarily functions in the kidneys to control phosphate homeostasis and active Vitamin D levels.

### SUMMARY OF THE INVENTION

The present invention provides compositions, systems, and methods for treating a condition characterized by elevated Fibroblast Growth Factor 23 (FGF23) with a composition comprising: i) an agent that causes an increase in expression of urokinase plasminogen activator (uPA) and/or tissue plasminogen activator (tPA), ii) purified uPA, and/or purified tPA, or derivatives thereof.

In some embodiments, provided herein are methods of treating a condition characterized by elevated Fibroblast Growth Factor 23 (FGF23) comprising: administering and/or prescribing at least one of the following to a subject with a condition characterized by elevated FGF23: i) a first composition comprising an agent that causes an increase in expression of a serine protease selected from urokinase plasminogen activator (uPA) and tissue plasminogen activator (tPA); and ii) a second composition comprising purified uPA, uPA derivative, tPA, and/or a tPA derivative.

In certain embodiments, the condition is selected from the group consisting of: chronic kidney disease, left ventricular hypertrophy and congestive heart failure, autosomal dominant hypophosphatemic ricketts, osteomalacia, Vitamin D deficiency, fibrous dysplasia, and aging. In some embodiments, the methods further comprise receiving a report or generating a report characterizing a biological sample from the subject as having elevated FGF23 levels compared to normal control population levels. In particular embodiments, the methods further comprise testing a sample from the subject to determine the level of FGF23 present in the sample.

In some embodiments, the agent that causes increase in expression of uPA and/or tTP comprises TM5441, which is shown in the following structure:

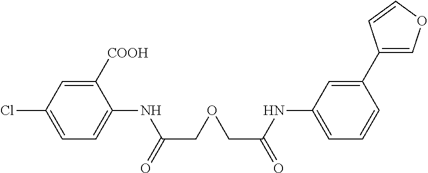 [SMILES: O=C(COCC(=O)Nc1ccc(Cl)cc1C(=O)O)Nc1cccc(-c2ccoc2)c1]

In some embodiments, the agent that causes increase in expression of uPA and/or tTP comprises TM5007, which is shown in the following structure:

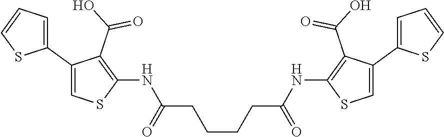 [SMILES: O=C(CCCCC(=O)Nc1scc(-c2cccs2)c1C(=O)O)Nc1scc(-c2cccs2)c1C(=O)O]

In some embodiments, the agent that causes increase in expression of uPA and/or tTP comprises TM5275, which is shown in the following structure:

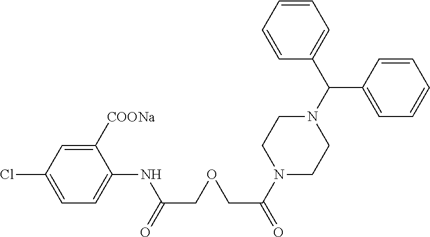

In certain embodiments, the subject is administered the first composition, wherein the agent comprises TM5441, TM5275, or TM5007, and wherein the TM5441, TM5275, or TM5007 is administered at a dose of 25-250 mg per kg of subject for at least 10 days (e.g., 10 days . . . 25 days . . . 100 days . . . 365 days . . . 500 days . . . 1000 days or more).

In particular embodiments, the subject is a human (e.g., human male or human female). In other embodiments, the subject is administered the first composition. In some embodiments, the subject is administered the second composition.

In particular embodiments, provided herein are systems comprising: a) a first component selected from: i) a first composition comprising an agent that causes an increase in expression of a serine protease selected from urokinase plasminogen activator (uPA) (e.g., human uPA) and tissue plasminogen activator (tPA) (e.g., human tPA); and ii) a second composition comprising purified uPA, uPA derivative, tPA, and/or tPA derivative; and b) a second component comprising a report characterizing a subject as having elevated levels of FGF23 and/or having a condition characterized by elevated Fibroblast Growth Factor 23 (FGF23).

In particular embodiments, the report is electronic or on paper. In other embodiments, the condition is selected from the group consisting of: chronic kidney disease, left ventricular hypertrophy and congestive heart failure, autosomal dominant hypophosphatemic ricketts, osteomalacia, Vitamin D deficiency, fibrous dysplasia, and aging. In additional embodiments, the report characterizes a biological sample from a subject as having elevated FGF23 levels compared to normal control population levels.

In certain embodiments, the subject is a human. In other embodiments, the first component comprises the first composition. In additional embodiments, the first composition comprises TM5441, TM5275, or TM5007. In other embodiments, the first component comprises the second composition.

In some embodiments, provided herein are systems comprising: a) a first component selected from: i) a first composition comprising an agent that causes an increase in expression of a serine protease selected from urokinase plasminogen activator (uPA) and tissue plasminogen activator (tPA); and ii) a second composition comprising purified uPA, uPA derivative, tPA, and/or tPA derivative; and b) a second component comprising components for testing a sample from a subject to detect FGF23 levels. In particular embodiments, the first component comprises the first composition, and wherein the first composition comprises TM5411.

In certain embodiments, provided herein are compositions comprising TM5441, TM5007, and/or TM5275 where the TM5441, TM5007, and/or TM5275 is present in said composition in therapeutically effective amounts for treating a subject (e.g., human) with a condition caused by elevated FGF23. In some embodiments, the composition further comprises a physiologically acceptable buffer, diluent, excipient, or the molecules. In particular embodiments, the composition is formulated in a pill, capsule, lotion gel, ointment, or in a patch.

### DESCRIPTION OF THE FIGURES

FIG. 1—FGF23 plasma levels in WT: wild type, kl/kl: klotho mice, kl/kl+TM5441: kl/kl mice treated with TM5441 for 120 days, kl/klpai-1^{-/-}: kl/kl mice lacking PAI-1, pai-1-stab: transgenic mice overexpressing human PAI-1, and pai-1^{-/-}: PAI-1 deficient mice. It is noted that PAI-1 is a serine protease inhibitor (serpin) that functions as the principal inhibitor of tissue plasminogen activator (tPA) and urokinase (uPA),

FIG. 2—Silver Stained SDS-PAGE gel of the in vitro FGF23 proteolysis by t-PA and uPA after 4 hours at 37° C.

FIG. 3—Silver Stained SDS-PAGE gel displaying the in vitro FGF23 proteolysis by t-PA after 4 hours at 37° C., which is inhibited by the presence of PAI-1 in the reaction. Proteolysis is then restored by the addition of TM5441.

FIG. 4—Western blotting of the in vitro FGF23 proteolysis by t-PA after various time points at 37° C. displaying the N-term and C-term fragments.

FIG. 5 shows the chemical structure of TM5441.

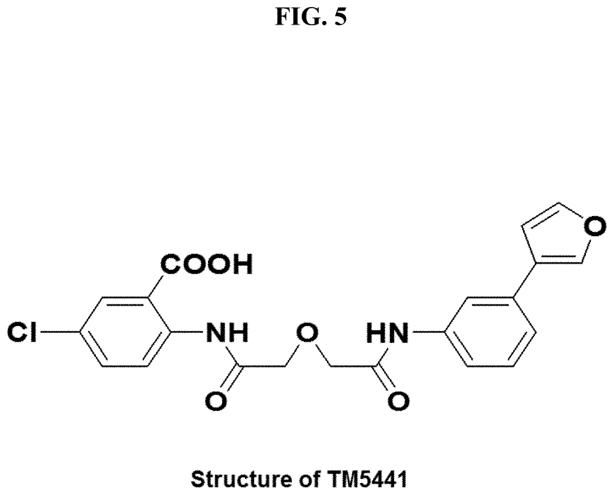

FIG. 6 shows the chemical structure of TM5007

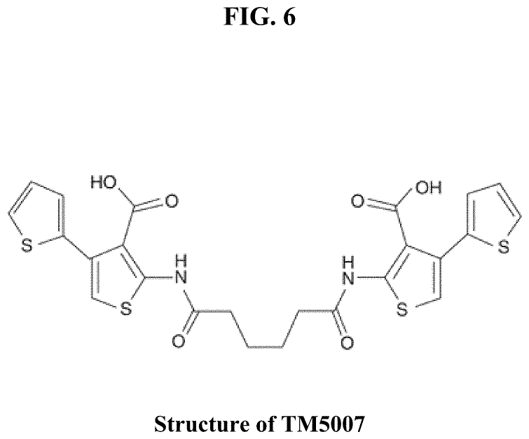

FIG. 7 shows the chemical structure of TM5275.

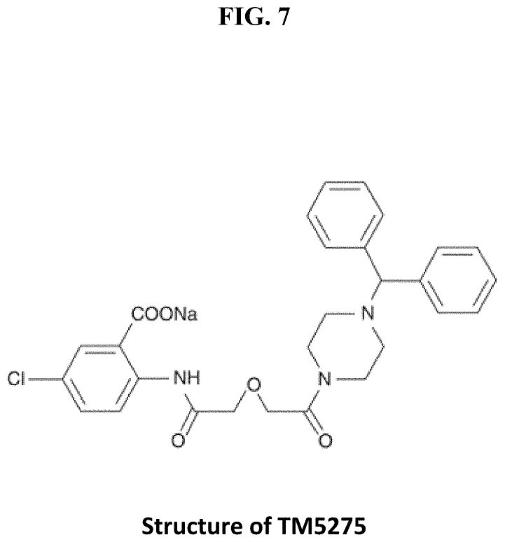

### DETAILED DESCRIPTION

The present invention provides compositions, systems, and methods for treating a condition characterized by elevated Fibroblast Growth Factor 23 (FGF23) with a composition comprising: i) an agent that causes an increase in expression of urokinase (uPA) and/or tissue plasminogen activator (tPA), ii) purified uPA, and/or purified tPA.

Elevated plasma levels of Fibroblast Growth Factor 23 (FGF23) are present in a variety of human diseases and conditions including chronic kidney disease, left ventricular hypertrophy and congestive heart failure, autosomal dominant hypophosphatemic ricketts, osteomalacia, Vitamin D deficiency, fibrous dysplasia, and aging. FGF23 is synthesized in bone by osteogenic cells, and is secreted into the circulation where it primarily functions in the kidneys to control phosphate homeostasis and active Vitamin D levels. In plasma, FGF23 can be cleaved between R179 and S180, yielding inactive N- and C-terminal fragments. It is speculated that a subtilisin-like proprotein convertase mediates the inactivating proteolysis of FGF23, but the specific protease(s) involved have not been identified until the present disclosure. The present disclosure reveals that two previously described proteases (uPA and tPA) can cleave FGF23, and methods that increase the activity of one or both of these proteases can reduce or normalize FGF23 levels in plasma.

Therefore, increasing the activity or molar amount of these serine proteases by any method in a subject will reduce the levels of FGF23. This strategy is attractive for the therapy of diseases that display elevated FGF23 levels since dosing can be modulated, for example, to maintain physiological levels of FGF23. In general, complete neutralization of FGF23 is not desirable since phosphate homeostasis and active Vitamin D levels need to be maintained.

In certain embodiments, urokinase plasminogen activator (uPA) sequence that is employed is human uPA, such as in SEQ ID NO:1 (Genback Accession No. CA26268) below, or a derivative (e.g., truncation or mutation thereof).

| (SEQ ID NO: 1) | |
|---|---|
| 1 | mrallarlll cvlvvsdskg snelhqvpsn cdclnggtcv snkyfsnihw cncpkkfggq |
| 61 | hceidksktc yegnghfyrg kastdtmgrp clpwnsatvl qqtyhahrsd alqlglgkhn |
| 121 | ycrnpdnrrr pwcyvqvglk plvqecmvhd cadgkkpssp peelkfqcgq ktlrprfkii |
| 181 | ggefttienq pwfaaiyrrh rggsvtyvcg gslmspcwvi sathcfidyp kkedyivylg |
| 241 | rsrlnsntqg emkfevenli lhkdysadtl ahhndiallk irskegrcaq psrtiqticl |
| 301 | psmyndpqfg tsceitgfgk enstdylype qlkmtvvkli shrecqqphy ygsevttkml |
| 361 | caadpqwktd scqgdsggpl vcslqgrmtl tgivswgrgc alkdkpgvyt rvshflpwir |
| 421 | shtkeengla l |

In some embodiments, a derivative of SEQ ID NO:1 is employed that shares at least 70% sequence identify with SEQ ID NO:1 (e.g., at least 70% . . . 80% . . . 90% . . . 95% . . . or 99% sequence identity). In certain embodiments, a truncated version of SEQ ID NO:1 is employed that is missing the N-terminal or C-terminal 10 . . . 20 . . . or 30 amino acids. In some embodiments, one, two, or three amino acids are mutated (e.g., conservative mutation) in SEQ ID NO:1, where the activity of the protein (e.g., cleaving FGF23) is not destroyed.

In other embodiments, the urokinase plasminogen activator (uPA) sequence that is employed is human uPA, such as in SEQ ID NO:2 (Genback Accession No. AAV70488) below, or a derivative (e.g., truncation or mutation thereof).

| (SEQ ID NO: 2) | |
|---|---|
| 1 | snelhqvpsn cdclnggtcv snkyfsnihw cncpkkfggq hceidksktc yegnghfyrg |
| 61 | kastdtmgrp clpwnsatvl qqtyhahrsd alqlglgkhn ycrnpdnrrr pwcyvqvglk |
| 121 | plvqecmvhd cadgkkpssp peelkfqcgq ktlrprfkii ggefttienq pwfaaiyrrh |
| 181 | rggsvtyvcg gslispcwvi sathcfidyp kkedyivylg rsrlnsntqg emkfevenli |
| 241 | lhkdysadtl ahhndiallk irskegrcaq psrtiqticl psmyndpqfg tsceitgfgk |
| 301 | enstdylype qlkmtvvkli shrecqqphy ygsevttkml caadpqwktd scqgdsggpl |
| 361 | vcslqgrmtl tgivswgrgc alkdkpgvyt rvshflpwir shtkeengla l. |

In some embodiments, a derivative of SEQ ID NO:2 is employed that shares at least 70% sequence identify with SEQ ID NO:2 (e.g., at least 70% . . . 80% . . . 90% . . . 95% . . . or 99% sequence identity). In certain embodiments, a truncated version of SEQ ID NO:2 is employed that is missing the N-terminal or C-terminal 10 . . . 20 . . . or 30 amino acids. In some embodiments, one, two, or three amino acids are mutated (e.g., conservative mutation) in SEQ ID NO:2, where the activity of the protein (e.g., cleaving FGF23) is not destroyed.

In certain embodiments, the tissue plasminogen activator (tPA) sequence that is employed is human tPA, such as in SEQ ID NO:3 (Genback Accession No. CAA00299) below, or a derivative (e.g., truncation or mutation thereof).

| (SEQ ID NO: 3) | |
|---|---|
| 1 | mdamkrglcc vlllcgavfv spsqeiharf rrgarsyqvi crdektqmiy qqhqswlrpv |
| 61 | lrsnrveycw cnsgraqchs vpvkscsepr cfnggtcqqa lyfsdfvcqc pegfagkcce |
| 121 | idtratcyed qgisyrgtws taesgaectn wnssalaqkp ysgrrpdair lglgnhnycr |
| 181 | npdrdskpwc yvfkagkyss efcstpacse gnsdcyfgng sayrgthslt esgasclpwn |
| 241 | smiligkvyt aqnpsaqalg lgkhnycrnp dgdakpwchv lknrrltwey cdvpscstcg |
| 301 | lrqysqpqfr ikgglfadia shpwqaaifa khrrspgerf lcggilissc wilsaahcfq |
| 361 | erfpphhltv ilgrtyrvvp geeeqkfeve kyivhkefdd dtydndiall qlksdssrca |
| 421 | qessvvrtvc lppadlqlpd wtecelsgyg khealspfys erlkeahvrl ypssrctsqh |
| 481 | llnrtvtdnm lcagdtrsgg pqanlhdacq gdsggplvcl ndgrmtlvgi iswglgcgqk |
| 541 | dvpgvytkvt nyldwirdnm rp. |

In some embodiments, a derivative of SEQ ID NO:3 is employed that shares at least 70% sequence identify with SEQ ID NO:3 (e.g., at least 70% . . . 80% . . . 90% . . . 95% . . . or 99% sequence identity). In certain embodiments, a truncated version of SEQ ID NO:3 is employed that is missing the N-terminal or C-terminal 10 . . . 20 . . . or 30 amino acids. In some embodiments, one, two, or three amino acids are mutated (e.g., conservative mutation) in SEQ ID NO:3, where the activity of the protein (e.g., cleaving FGF23) is not destroyed.

In certain embodiments, the tissue plasminogen activator (tPA) sequence that is employed is human tPA, such as in SEQ ID NO:4 (Genback Accession No. BAA00881) below, or a derivative (e.g., truncation or mutation thereof).

| | |
|---|---|
| 1 | rrgarsyqvi crdektqmiy qqhqswlrpv lrsnrveycw cnsgraqchs vpvkscsepr |
| 61 | cfnggtcqqa lyfsdfvcqc pegfagkcce idtratcyed qgisyrgtws taesgaectn |
| 121 | wnssalaqkp ysgrrpdair lglgnhnycr npdrdskpwc yvfkagkyss efcstpacse |
| 181 | gnsdcyfgng sayrgthslt esgasclpwn smiligkvyt aqnpsaqalg lgkhnycrnp |
| 241 | dgdakpwchv lknrrltwey cdvpscstcg lrqysqpqfr ikgglfadia shpwqaaifa |
| 301 | khrrspgerf lcggilissc wilsaahcfq erfpphhltv ilgrtyrvvp geeeqkfeve |
| 361 | kyivhkefdd dtydndiall qlksdssrca qessvvrtvc lppadlqlpd wtecelsgyg |
| 421 | khealspfys erlkeahvrl ypssrctsqh llnrtvtdnm lcagdtrsgg pqanlhdacq |
| 481 | gdsggplycl ndgrmtlvgi iswglgcgqk dvpgvytkvt nyldwirdnm rp |

In some embodiments, a derivative of SEQ ID NO:4 is employed that shares at least 70% sequence identify with SEQ ID NO:4 (e.g., at least 70% . . . 80% . . . 90% . . . 95% . . . or 99% sequence identity). In certain embodiments, a truncated version of SEQ ID NO:4 is employed that is missing the N-terminal or C-terminal 10 . . . 20 . . . or 30 amino acids. In some embodiments, one, two, or three amino acids are mutated (e.g., conservative mutation) in SEQ ID NO:4, where the activity of the protein (e.g., cleaving FGF23) is not destroyed.

In particular embodiments, the tissue plasminogen activator (tPA) sequence that is employed is human tPA, such as shown in Genbank Accession numbers AA034406, CAX11668, or CAA00642 (all of which are herein incorporated by reference). In some embodiments, a derivative of one of these three sequences is employed that shares at least 70% sequence identify with one of these sequences (e.g., at least 70% . . . 80% . . . 90% . . . 95% . . . or 99% sequence identity). In certain embodiments, a truncated version of one of these sequences is employed that is missing the N-terminal or C-terminal 10 . . . 20 . . . or 30 amino acids. In some embodiments, one, two, or three amino acids are mutated (e.g., conservative mutation) in one of these sequences, where the activity of the protein (e.g., cleaving FGF23) is not destroyed.

### EXAMPLES

### Example 1

### Serine Proteases Urokinase (uPA) and Tissue Plasminogen Activator (tPA) Cleave FGF23

This Example describes the ability of uPA and tPA to cleave FGF23.

### Materials and Methods

### Recombinant Proteins:

Recombinant human t-PA, uPA, and stable PAI-1 were obtained from Molecular Innovations Inc., Novi, Mich. Recombinant Human FGF23 was obtained from R and D Systems Inc., Minneapolis Minn.

### Tm5441:

TM5441 was obtained from Toshio Miyata, M. D., Ph.D. and Renascience, Inc. A stock solution of 2.5 mM was prepared in DMSO.

### Western Blotting:

The 6×His tag antibody was obtained from Abcam Inc., Cambridge Mass., and the FGF23 antibody was obtained from Santa Cruz Biotechnology Inc., Dallas Tex. Secondary HRP conjugated donkey anti goat (cross-adsorbed) and goat anti-rabbit antibodies were obtained from Bethyl Laboratories Inc., Montgomery, Tex., and Molecular Innovations Inc. respectively. 4-12% NuPAGE Bis-Tris Gels, PVDF iblot gel transfer stacks, 4×NuPAGE LDS sample buffer and 10×NuPAGE sample reducing agent were from Life Technologies Inc., Carlsbad, Calif. Luminata Forte Western HRP Substrate detection reagent was from EMD Millipore Inc., Billerica, Mass.

### Silver Staining of SDS-PAGE Gels:

Gels were silver stained using the Pierce Silver Stain Kit from Thermo Fisher Scientific Inc. Waltham, Mass., according to the manufacturer's protocol.

### In Vitro Protease Reactions:

Recombinant human FGF23 (1-1.5 μg) was incubated at 37° C. for 4 hours (or other time point indicated) with either recombinant human t-PA (1.1 μg) or uPA (1.1 μg), (in addition to 1.70 μg of recombinant human stable PAI-1 where indicated) in reaction buffer (25 μl final volume) containing: 50 mM Tris-HCl pH 8.0, 150 mM NaCl, 200 mM CHAPS, 0.1% PEG-8000, and 1% DMSO. To terminate the reaction, samples were incubated at 95° C. for 5 minutes in the presence of 1×NuPAGE LDS sample buffer and 1× NuPAGE sample reducing agent.

### TM5441 Treatment of Klotho Mice:

Four week old Klotho (kl/kl) mice (n=11) were administered TM5441 at the dose of 100 mg/kg/day for 120 days. The chow diet containing the inhibitor was prepared by mixing a homogenous suspension of TM5441 made in 0.5% Carboxy Methyl Cellulose into the powder chow diet (Harland Teklad LM-485 number 7012). The plasma levels of FGF23 was measured using an ELISA kit (Immutopics, Inc. Mouse/Rat FGF-23 (C-Term) ELISA Kit, Cat. #60-6300) by following the manufacturer's protocol.

### Results:

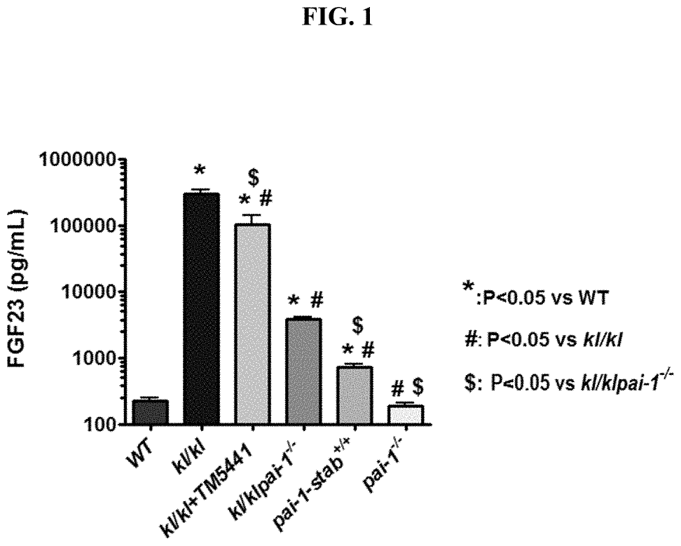

In a mouse model with extremely elevated FGF23 levels (Klotho mice), FGF23 levels are markedly reduced by treatment with TM5441, a small orally-active molecule that increases t-PA and uPA activity. FIG. 1 shows the results, which further include mice lacking or over-expressing PAI-1, a known inhibitor of t-PA and uPA.

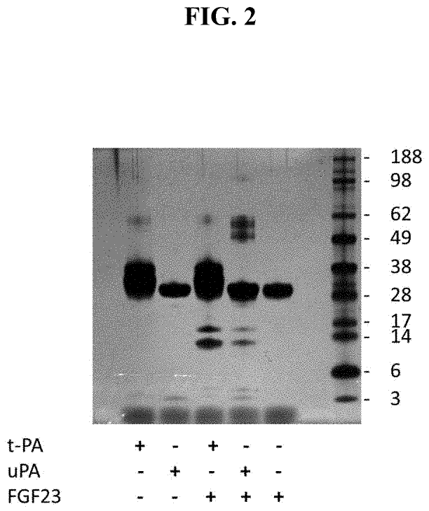

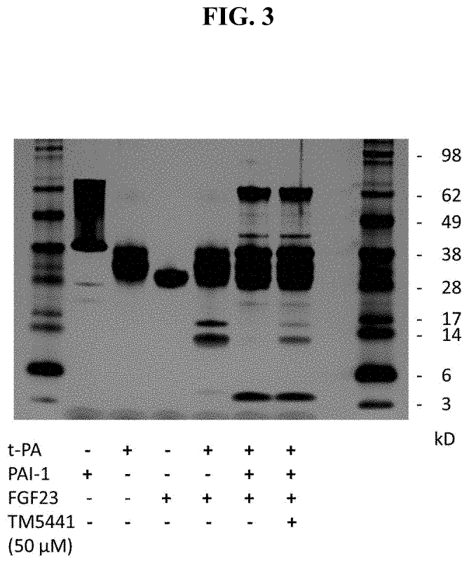

When recombinant purified human FGF23 is exposed to recombinant purified human t-PA or uPA serine proteases, smaller fragments are generated on a silver stained SDS-PAGE gel indicating proteolytic cleavage. These results are shown in FIG. 2. FIG. 3 further shows silver stained SDS-PAGE gel displaying the in vitro FGF23 proteolysis by t-PA after 4 hours at 37° C., which is inhibited by the presence of PAI-1 in the reaction. Proteolysis is then restored by the addition of TM5441.

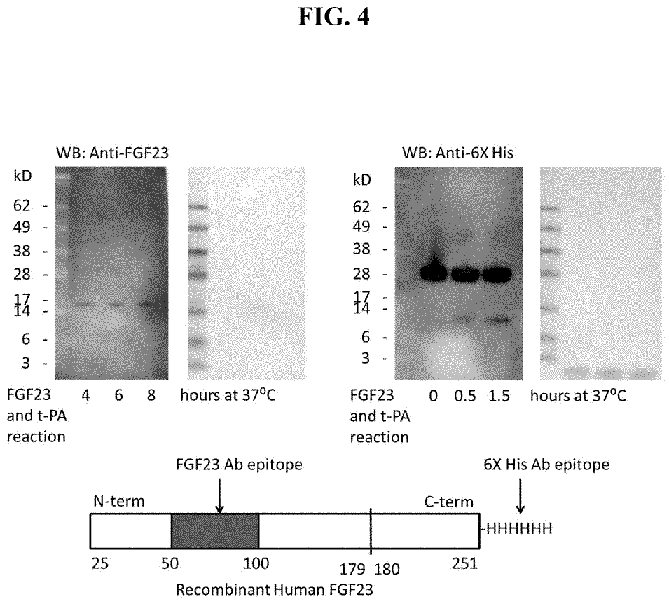

The smaller fragments are confirmed to be of FGF23 origin by western blot as well. This is shown in FIG. 4, which provides a Western blot of the in vitro FGF23 proteolysis by t-PA after various time points at 37° C. displaying the N-term and C-term fragments.

These data indicate that FGF23 proteolytic metabolism yielding inactive fragments is mediated, at least in part, by uPA and t-PA in vitro and in vivo. Thus increasing uPA or t-PA activity or molar amount, will result in reduced or normalized active FGF23 levels in vivo.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a condition characterized by elevated Fibroblast Growth Factor 23 (FGF23) comprising:
administering and/or prescribing at least one of the following to a subject with a condition characterized by elevated FGF23:
i) a first composition comprising an agent that causes an increase in expression of a serine protease selected from urokinase plasminogen activator (uPA) and tissue plasminogen activator (tPA); and
ii) a second composition comprising purified uPA, uPA derivative, tPA, and/or a tPA derivative.

2. The method of claim 1, wherein said condition is selected from the group consisting of: chronic kidney disease, left ventricular hypertrophy and congestive heart failure, autosomal dominant hypophosphatemic ricketts, osteomalacia, Vitamin D deficiency, fibrous dysplasia, and aging.

3. The method of claim 1, further comprising receiving a report or generating a report characterizing a biological sample from said subject as having elevated FGF23 levels compared to normal control population levels.

4. The method of claim 1, further comprising: testing a sample from said subject to determine the level of FGF23 present in said sample.

5. The method of claim 1, wherein said agent comprises TM5441, TM5275, or TM5007.

6. The method of claim 1, wherein said subject is administered said first composition, wherein said agent comprises TM5441, TM5275, or TM5007, and wherein said TM5441, TM5275, or TM5007 is administered at a dose of 25-250 mg per kg of subject for at least 10 days.

7. The method of claim 6, wherein said at least 10 days is at least 100 days.

8. The method of claim 1, wherein said subject is a human.

9. The method of claim 1, wherein said subject is administered said first composition.

10. The method of claim 1, wherein said subject is administered said second composition.

11. A system comprising:
a) a first component selected from:
i) a first composition comprising an agent that causes an increase in expression of a serine protease selected from urokinase plasminogen activator (uPA) and tissue plasminogen activator (tPA); and
ii) a second composition comprising purified uPA, uPA derivative, tPA, and/or tPA derivative; and
b) a second component comprising a report characterizing a subject as having elevated levels of FGF23 and/or having a condition characterized by elevated Fibroblast Growth Factor 23 (FGF23).

12. The system of claim 11, wherein said report is electronic or on paper.

13. The system of claim 11, wherein said condition is selected from the group consisting of: chronic kidney disease, left ventricular hypertrophy and congestive heart failure, autosomal dominant hypophosphatemic ricketts, osteomalacia, Vitamin D deficiency, fibrous dysplasia, and aging.

14. The system of claim 11, wherein said report characterizes a biological sample from a subject as having elevated FGF23 levels compared to normal control population levels.

15. The system of claim 11, wherein said subject is a human.

16. The system of claim 11, wherein said first component comprises said first composition.

17. The system of claim 16, wherein said first composition comprises TM5441.

18. The system of claim 11, wherein said first component comprises said second composition.

19. A system comprising:
a) a first component selected from:
i) a first composition comprising an agent that causes an increase in expression of a serine protease selected from urokinase plasminogen activator (uPA) and tissue plasminogen activator (tPA); and
ii) a second composition comprising purified uPA, uPA derivative, tPA, and/or tPA derivative; and
b) a second component comprising components for testing a sample from a subject to detect FGF23 levels.

20. The system of claim 19, wherein said first component comprises said first composition, and wherein said first composition comprises TM5411, TM5275, or TM5007.
